(19) United States
(12) Patent Application Publication
(10) Pub. No.: US 2010/0076386 A1
(43) Pub. Date: Mar. 25, 2010

(54) Method and Apparatus to Induce Detachment of Flagella Propelled Micro Organisms from Walls and to Countermeasure Their Ability to Swim Upstream

(76) Inventor: Guillermo Vidal-de-Miguel, Valladolid (ES)

Correspondence Address: Guillermo Vidal-de-Miguel, Calle de los Cipreses N 18, Madrid, Madrid, 28036, ES

(21) Appl. No.: 12/563,018

(22) Filed: Sep. 18, 2009

Related U.S. Application Data
(60) Provisional application No. 61/192,682, filed on Sep. 22, 2008.

(51) Int. Cl. A61M 25/00; C12M 1/00

(52) U.S. Cl. 604/264; 435/283.1

(57) ABSTRACT

Medical catheters and catheter drainages can be difficult to disinfect. Infections produced by microorganisms swimming upstream through said catheters and catheter drainages are thus difficult to eliminate.

A method to detach flagella propelled microorganisms from walls and to countermeasure their ability to swim upstream against the velocity of the fluid in ducts is described. Observations show that flagella propelled microorganisms require the forces and torques induced by the proximity of a wall and also by a velocity gradient produced near the wall of said ducts. The method is based on detaching said flagella propelled microorganisms from the walls by means of sharp edges. The flagella propelled microorganism can not change its swimming direction so abruptly to follow the geometry of said sharp edge and therefore it detaches from the wall. Once said microorganism is detached from the wall, it is flushed downstream by the velocity of the fluid which is stronger once far from the wall. As a result, flagella propelled microorganisms are prevented from swimming upstream.

### CROSS REFERENCE TO RELATED APPLICATION

This application claims the benefit of priority to U.S. Provisional Patent Application No. US61/192,682, filed on Sep. 22, 2008.

### U.S. PATENT DOCUMENTS CITED

“Not Applicable”

### OTHER PUBLICATIONS CITED

[1] Journal of bacteriology; F. Garcia-Pichel; Rapid Bacterial Swimming Measured in Swarming Cells of Thiovulum majus; Vol. 171, No. 6, p. 3560-3563; June 1989,
[2] Physical Review Letters; Hill J., Kalkanci O., L. McMurry J., Koser H.; Hydrodynamic Surface Interactions Enable Escherichia Coli to Seek Efficient Routes to Swim Upstream; PRL 98, 068101; 9 Feb. 2007
[3] Biophysics, Proc. Natl. Acad. Sci. USA; Robert M. Macnab; Bacterial Flagella Rotating In Bundles: A Study In Helical Geometry; Vol. 74, No. 1, pp. 221-225; Oct. 29, 1976.
[4] Biophys J BioFAST; Lauga E., DiLucio W. R., Whitesides G. H. Stone H. A., Swimming In Circles: Motion Of Bacteria Near Solid Boundaries; doi:10.1529/biophysj.105.069401; Oct. 20, 2005
[5] Applied And Environmental Microbiology; Margot A. S. Vigeant, Roseanne M. Ford, Michael Wagner, and Lukas K. Tamm; Reversible and Irreversible Adhesion of Motile Escherichia coli Cells Analyzed by Total Internal Reflection Aqueous Fluorescence Microscopy; Vol. 68, No. 6, p. 2794-2801; Mar. 20, 2002
[6] Biophysics, Proc. Natl. Acad. Sci. USA; Paul D. Frymier, Roseanne M. Ford, Howard C. Bergo, And Peter T. Cummings; Three-Dimensional Tracking Of Motile Bacteria Near A Solid Planar Surface; Vol. 92, pp. 6195-6199; Mar. 29, 1995
[7] Journal Of Bacteriology; Peter Galajda, Juan Keymer, Paul Chaikin, and Robert Austin; A Wall of Funnels Concentrates Swimming Bacteria; Vol. 189, No. 23, p. 8704-8707; Aug. 31, 2007

### FIELD OF THE INVENTION

Driving fluids in the presence of flagella propelled microorganisms as E-Coli bacteria or spermatozoids can be a problem in certain applications in which it is desirable to avoid the migration of said microorganisms.

Any microorganism is capable of migrating downstream the flow. That is easy since they can be passively transported by the fluid velocity. If this was the only mean of migration, it would be possible to ensure that the upstream of the flow can be free of microorganisms coming from downstream relying only on fluid dynamics of normal tubes. But, unfortunately, flagella propelled microorganism are capable of swimming upstream the flow using the walls of the tubes.

Microorganisms can be eliminated by several aggressive means such as aggressive chemical agents, extreme temperatures or radiations.

This invention relates to an apparatus and a method for driving fluids while counter measuring the ability of flagella propelled microorganisms to swim upstream of the flow. This is done relying on a specific geometric design of the walls of the ducts in order to modify the fluid mechanics of the liquid driven by the duct and the microorganism swimming mechanics. The invention prevents microorganisms from migrating upstream and it is non aggressive with neither the microorganisms nor the fluid itself.

### BACKGROUND OF THE INVENTION

The related problem of (i) driving a fluid online through a duct form an origin to a destination while (ii) assuring that no microorganisms will swim upstream has been of crucial interest. For instance, a catheter or a catheter drainage placed after a surgery in a human being can be understood as a system comprising an origin (the human body), a duct (the catheter or the catheter drainage) and a destination (the sink). In this case, the destination could be contaminated since it is opened to the environment. And it is of crucial interest to maintain microorganism away from the human body. This kind of problem can be solved by different means such as using chemical disinfectants or extreme temperatures. Other methods comprise exposure to radiations. Those methods of disinfection comprise destroying and killing the microorganisms. Those methods of disinfection can also destroy or modify complex molecules such as proteins. For these reasons, said methods are termed herein aggressive to the microorganisms. On the other hand, adding chemical disinfectant changes the chemical properties of the fluid. For this reason, said chemical disinfection methods are also termed herein aggressive to the fluid. In general, disinfectants are also harmful for the human or other living organisms. Though important efforts have lead to lesser toxic disinfectants, effective chemical disinfectants are still toxic to a greater or lesser extent. Thus, the disinfection of the fluid by means of chemical disinfection also comprises that the fluid will no longer be safe for life when it arrives at the destination.

Microorganisms can be classified according to their motility. Though many mechanisms remain unknown or only partially understood, the main mechanisms observed are flagella action, cilia action, pseudopodia, which are membrane covered cytoplasm extensions, and gliding motility. Among the different types of motility mechanism, the flagellum motility is of special interest for two reasons. First, because it provides the microorganisms with a considerable speed which is typically around 50 μm/s for fast flagellated bacteria, 200 μm/s for fast flagellated protozoa and 50 μm/s for spermatozoids [1]. And second, because, as noted by Hill et al [2], this type of microorganisms are capable of swimming heading upstream with a steady direction under the influence of the wall and the fluid velocity gradient produced near the walls of the duct when the fluid is flowing through it. This feature gives these flagellated propelled microorganisms (From now on termed as FPMO) an extraordinary capacity of migrating upstream when compared with other microorganisms. In normal conditions, FPMO can travel up to 6 mm in one minute at a speed of 100 microns/s.

Among the ranking of the fastest microorganisms [1], the fastest known flagellated bacteria (Thiovulum majus) can swim at 600 μm/s; and the fastest known microorganism is a ciliated paramecium which can swim at 1000 μm/s. Though said paramecium is very fast, it does not have the ability to face steadily upstream. No matter how fast they swim, microorganisms located in the regions where the fluid velocity is high enough will be irremediably flushed downstream by the fluid velocity. For this reason, only those microorganisms that remain steadily in the vicinity of the walls, where the fluid velocity is very low, can swim upstream. But, once in the vicinity of the wall, if the microorganism chooses its velocity randomly or pseudo-randomly, its net upstream velocity become statistically zero and those microorganisms become very unlikely to travel effectively upstream. This can be measured by the probability that a microorganism travels all along the duct and reaches the origin, which will be termed P from now on. If the microorganism follows a random trajectory, P decreases very rapidly as the duct is made longer. But, as shown by Hill et al [2], FPMO can swim very high distances always heading upstream in a stable direction. In this case, using longer ducts is not an effective mean to reduce P since it only makes them to take longer times to arrive at the origin. In practice, only FPMO are really capable of traveling upstream through relatively long ducts. The present invention is therefore specially focused on counter-measuring ability of FPMO to swim upstream.

FPMO Free Swimming.

A FPMO is a microorganism that uses flagella for translation. A FPMO is composed by a body, which is bounded by the cell wall, and the flagella. FPMO have one single flagellum or more than one. When the FPMO having more than one flagellum rotates its flagellums, they form a bundle that propels the cell forward. Surprisingly, said bundle behaves mechanically smoothly. The behavior of the bundle has been studied in detail by Robert M. Macnab [3]. One of his conclusions is that the bundle behaves as one single entity producing a trust and a torque as well as the flagellum. Typical examples of FPMO are E-coli bacterium and spermatozoids. Escherichia coli cells use long, thin structures called flagella to propel themselves. These flagella form a bundle that rotates counter-clockwise, creating a torque that causes the bacterium to rotate clockwise. Spermatozoids are a classic example of FPMO having one single flagellum. FIG. 1 illustrates the typical structure of an FPMO comprising at least a body (1) and at least one flagellum, or a bundle formed by more than one flagellum. For simplicity, in FIG. 1 the bundle or the single flagellum is drawn schematically as one single flagellum (2). Also for simplicity, as the single flagellum and the bundle behave similarly in terms of the FPMO swimming mechanics [3], only the term flagellum will be used from now on referred to either the bundle or a single flagellum.

Typical Reynolds of the body of a FPMO with a length of a few microns and swimming at a speed of 100 microns/s in water (8.9·10⁻⁴ Pa⁻¹ viscosity; 1000 Kg/m³ density) is of the order 10⁻⁴ (Far below from zero). This implies that viscous forces govern the movement. As the density of the FPMO is similar to water, its inertia will be much smaller than the viscous forces and can be also neglected in the equations of its movement while swimming. These considerations are especially useful to analyze the movement of the FPMO. As inertia can be always neglected, the equations of the movement are governed only by the equilibrium of induced forces and torques. This low Reynolds regime is usually termed force free torque free swimming.

Experimental observations show that, when the FPOM are swimming freely in unbounded fluids, they usually describe sequences of runs and turns. During a run, the FPMO swims in a straight trajectory. During a turn, the FPMO changes the rotation direction of at least one of its flagellum resulting in a change of direction rather than a proper displacement.

When moving straight, the FPMO rotates its flagella in a direction of rotation. The torque produced by the viscous fluid on the rotating flagellum is in the same direction of the rotation and is compensated by the torque produced by the body which rotates in the opposite direction. The rotation of the flagellum also produces a trust in the direction of rotation; this can be better visualized considering the bundle as a screw in viscous flow. This trust is compensated by the drag acting on the body and on the flagellum of the FPMO caused by a velocity in the same direction of the trust force produced by flagellum. The equilibrium of forces and momentum is achieved and the net result of the flagellum rotation is a body rotation in the opposite direction and a net velocity in the direction opposite to the flagellum rotation, all in the same axis. The net effect is a straight trajectory of the FPMO, also called a ‘run’. The FIG. 2 shows a scheme of a FPMO swimming in the absence of obstacles and velocity gradients with basic forces and torques. Trust (3) and axial drag (4) forces are counterbalanced. Axial torque (5) produced by the rotating bundle (2) and axial torque (6) produced by the counter-rotating body (1) are also counterbalanced. Note that, as previously explained, the inertia has been neglected.

FPMO Swimming Near a Wall with the Flow at Rest

Experimental observations show that, when the FPOM are swimming very near to the wall and with the flow at rest, they usually describe circular trajectories for several minutes as if they were trapped by the wall [4, 5, 6]. Note that, though the FPMO can not leave the wall, they are not stuck on the wall since they can move. The attraction effect induced by the proximity of the wall is still not fully understood. However, there are evidences that demonstrate that this attraction is governed mainly by hydrodynamic effects [5] produced by the interaction of rotating parts of the FPMO and the wall. For this reason, said interaction is termed hydrodynamic trapping.

FIG. 3 shows all the angles, axes and coordinates required to describe in a first approximation how the FPMO behaves near a wall. The orthogonal axes x, y and z are used. The x, y and z axes (represented respectively with references 7, 8 and 9 in FIG. 3) are solid with the wall. In these axes the position of the wall can be described as z=0, and the velocity of the wall is zero.

In a first approximation, the FPMO will be treated geometrically as a segment that can be described by the direction of rotation of its flagellum (10) and the coordinates of one point of the axis of rotation. The direction of rotation of the flagellum (10) is herein described by the Euler angles θ (11) and δ (12). The angle δ is the angle between the axis of rotation of the flagellum (10) and the wall. The angle θ is the angle formed between the projection of the axis of rotation of the flagellum (10) on the wall and the x axis (7). The point of the axis of rotation required to complete the simplified geometrical description the FPMO is chosen arbitrarily such that when the FPMO is swimming near a wall, its z coordinate is equal to zero.

The kinematics of the simplified FPMO can be fully described with the velocity of said point of the axis of rotation and the time derivatives of the angles θ (11) and δ (12). As the FPMO remains adjacent to the wall, its velocity vector is parallel to the wall. In such conditions, the velocity vector can be described by its magnitude v (13) and the angle α (14). The angle α is herein the angle formed between said velocity vector and the y axis. FIG. 3 also shows the typical position adopted by the FPMO when swimming under the influence of the wall aiming at the wall with an angle δ between 0 and 90 degree.

The reason why the FPOM swims in circles can be explained if we assume that the interaction between the FPMO, and the wall produces, somehow, a torque on the FPMO that is perpendicular to the wall. This torque is counterbalanced by a movement associated with dθ/dt (denoting an angular velocity in the angle θ) [4]. Note again that inertia has been neglected. It is also very noticeable that the angle δ observed is between 0° and 90° [2]. This makes the FPMO to aim at the wall. If a perturbation separates slightly the FPMO from the wall, the FPMO will return to the wall as it is aiming towards it. This makes the configuration especially stable. The net result of the model proposed herein for a FPMO swimming near a wall with the flow at rest is that the FPMO aims at the wall swimming at a constant speed with a constant angular velocity perpendicular to the wall. The composition of a constant speed and said angular velocity is the circular trajectory already observed. As the configuration is stable, the FPMO seems to be trapped near the wall by the hydrodynamic trapping effect. A more extended model with similar conclusions and reasonable agreement with experimental data was been developed by Lauga et al [4].

FPMO Swimming Near a Wall with Moving Fluid Flow

The velocity of the flow does not have any effect by itself apart from carrying the FPMO downstream with the flow through the streamlines. Once the FPMO is moving with the flow it will behave as if the flow were at rest. It is a simple case of Galileans relativity since inertia can be neglected. What can definitely affect the FPMO swimming mechanics are the velocity gradients which can cause torques in the FPMO. Torques can be induced by velocity gradients because different parts of the FPMO are affected by different velocities and hence by different drags.

Close to a wall, in the area affecting a FPMO, when the flow is not at rest, the velocity profile starts with zero on the wall and grows as the distance to the wall (z in the coordinates system herein used) is higher. Locally, the velocity vector will have the same direction though its magnitude changes. In order to simplify the explanation, though in a general case the velocity gradient is a tensor that depends on the position, and more specifically on the z coordinate, only a the scalar value ∂|V_{f}|/∂z (where V_{f} is the fluid velocity vector) at z=0 will be considered herein.

In order to describe the FPMO behavior, the same system of coordinates used in FIG. 3 will be used herein. The direction of the fluid velocity is parallel to the x axis and the fluid is moving towards the positive side of the x axis.

The observed behavior of E-Coli [2] demonstrates that, surprisingly, under the effect of the velocity gradient, the bacteria no longer swims in circles, but it swims in a straight trajectory with a stable angle θ and a stable angle α rightwards and towards the upstream direction) (0°<α<90°). This behavior can be easily visualized as if the FPMO was a sail beating towards the wind. Swimming mechanics of any other FPMO is, in essence, the same as for E-Coli and their behavior is, therefore, to swim upstream at stable angles θ and α when they are influenced by the wall and the velocity gradient. Indeed, this behavior can be again explained by a simplified model assuming that the interaction between the FPMO and the wall produces a torque on the FPMO that is perpendicular to the wall. Said torque is based on the same premises as the torque produced when the fluid is at rest. Experimental observations also show that, for medium velocity gradients, between 1 s⁻¹ and 10 s⁻¹ for the case of E-Coli [2], the FPMO exhibits a position with its body aiming at the wall and its rotating flagellum standing up at a positive angle δ. In this configuration, the tale of the flagellum is under a higher drag in the direction of the velocity of the flow. The net effects of the velocity gradient are: (i) A torque perpendicular to the wall surface that tends to reduce |θ| and that is zero when the fluid velocity and the projection of the flagellum on the wall are aligned (when θ is zero or 180°). This torque makes the FPMO behave like a weathercock. (ii) A torque that tends to reduce the value of the angle δ when the FPMO swims against the flow (heading upstream) and that tends to increase δ when the FPMO swims with the flow (heading downstream). (iii) And a drag parallel to the velocity of the local flow.

Interestingly, at the observed angle δ, the velocity gradient is capable of producing an induced torque, termed herein T that counterbalances the torque induced by the proximity of the wall.

If the velocity gradient is strong enough, there will by two values of the angle θ at which the torque induced by the proximity of the wall is compensated solely with the torque T and therefore no angular velocity is required to counterbalance the torque induced by the wall. Only the angle at which the FPMO swims upstream is stable. When the FPMO swims upstream with said angle of equilibrium, a perturbation increasing the angle theta increases the torque that tends to reduce the perturbation, and therefore the FPMO returns to its equilibrium position. This is the weathercock effect. This weathercock effect makes the FPMO to position itself steadily towards the velocity flow. The net effect is that, under the effects of the wall and the velocity gradient, the FPMO swims upstream steadily with angles θ and α of equilibrium or turns smoothly until reaching said angles. Note again that inertia has been neglected. This explains why, under the effect of a velocity gradient, the FPMO stop swimming in circles on the wall and swims upstream in straight trajectories with said angles θ and α of equilibrium.

Observations also show that, when the velocity gradient is very high, for instance, higher than 10 s⁻¹ for the case of E-Coli [2], the swimming configuration changes. Under said velocity gradients, E-Coli starts drifting rightwards and downstream (180°<α<270°). The mechanics of this behavior remains unknown for the author of this invention. However, the conclusion is that sufficiently strong velocity gradients can prevent FPMO from swimming upstream.

Note also that, though the flow in the tube was turbulent due to a high Reynolds configuration, the FPMO would still have the ability to swim upstream as the lower layer of the fluid boundary layer remains laminar and the mechanics explained and observed would still apply.

A typical device of practical use in which it can be easy for a FPMO to swim upstream is a tube. If the radius or the tube is significantly bigger than the length of the FPMO, the FPMO behave locally as if the wall where plane and it swims heading upstream at its angles θ and α of equilibrium once it reaches the wall. The curvature of the wall produces a helical trajectory and the FPMO swims upstream like a screw moving forward upstream. The FIG. 4 shows a scheme of a FPMO swimming upstream in a tube. FIG. 4 shows a magnified FPMO (15) swimming under the influence of the inner wall (16) of the tube (17) and the velocity gradient, the helical trajectory (18) described by the FPMO (15), the velocity of the FPMO (13), and the direction of the flow (19). Note that the pitch angle of the helix is given by the angle α (14).

The purpose of the present invention is to find a solution to the related problem of (i) driving a fluid online through a duct form an origin to a destination while (ii) assuring that microorganisms will not swim upstream through said duct and also assuring that (iii) microorganisms and complex molecules traveling downstream with the fluid will not be destroyed, modified or killed; and (iv) that the chemical composition and properties of the fluid will remain unchanged. To achieve this goal, the method has to be non aggressive with either the fluid nor the microorganisms and the complex molecules.

### SUMMARY OF THE INVENTION

This invention is a way of detaching flagella propelled microorganisms from walls and to countermeasure their ability to swim upstream against the fluid velocity in ducts. Disinfection methods also prevent microorganisms from swimming upstream by killing them, but they can be too aggressive for certain applications. The present invention relies solely on a mechanical configuration and thus it is non aggressive. The present invention is especially interesting for those applications where it is desired to prevent microorganisms from swimming upstream while it is also needed to avoid aggressive techniques.

Observations show that flagella propelled microorganisms require the forces and torques induced by the proximity of a wall and also by a sufficient but not too strong velocity gradient. Once said microorganism is in the vicinity of the wall, it will swim heading steadily upstream trapped near the wall. If the duct is a cylindrical tube, said microorganisms will swim upstream describing a helix in the inner surface of said tube.

In the present invention, sharp edges are used to produce high velocity gradients and also to detach said microorganisms. The flagella propelled microorganism can not change its swimming direction so abruptly to follow the geometry of said sharp edge and therefore it detaches from the wall. Once said microorganism is detached from the wall, it is flushed downstream by the velocity of the fluid which is stronger once far from the wall. The sharp edge also produces a singular velocity gradient with which said microorganism can not swim steadily upstream. Any of those two effects is sufficient to prevent the microorganism from swimming upstream. In order to drive said microorganisms towards said sharp edge, it is al so necessary a geometry with a smooth transition between the original wall and the shard edge. The geometry comprising said smooth transition and said sharp edge is termed herein a ‘FPMO detachment sharp edge’. The trajectory of a flagella propelled microorganism in a tube comprising at least one FPMO detachment sharp edge will be a helix until, eventually, said microorganism reaches the sharp edge and it is flushed downstream. As a result, microorganisms can not swim upstream through said tube comprising at least one FPMO detachment sharp edge.

### BRIEF DESCRIPTION OF THE DRAWINGS

FIG. 1 illustrates schematically a flagella propelled microorganism (termed herein FPMO) comprising a flagella and a body.

FIG. 2 illustrates schematically the forces and torques induced by the fluid on the FPMO when the FPMO is swimming freely in unbounded fluid.

FIG. 3 illustrates the coordinates, the angles and the velocity vector used to describe the movement of the FPMO when it is swimming near a wall

FIG. 4 illustrates schematically a FPMO swimming upstream in a cylindrical tube. Note that the size of the FPMO has been magnified and is not realistic when compared with the size of the tube.

FIG. 5 illustrates schematically a section of a recirculation bubble and a reattachment line which acts as a barrier against FPMO

FIG. 6 illustrates schematically a section of a FPMO detachment sharp edge comprising a sharp edge and a smooth transition between the wall and said sharp edge FIG. 6 also illustrates a FPMO, and its trajectory which is detached from the wall.

FIG. 7 illustrates schematically a FPMO detachment sharp edge aligned with the flow, the different fluid velocity profiles, a FPMO and its trajectory.

FIG. 8 illustrates schematically a FPMO detachment tube comprising a FPMO detachment sharp edge. FIG. 8 also illustrates a FPMO, and its trajectory in said FPMO detachment tube.

FIG. 9 illustrates schematically a FPMO detachment tube comprising standard tube fittings.

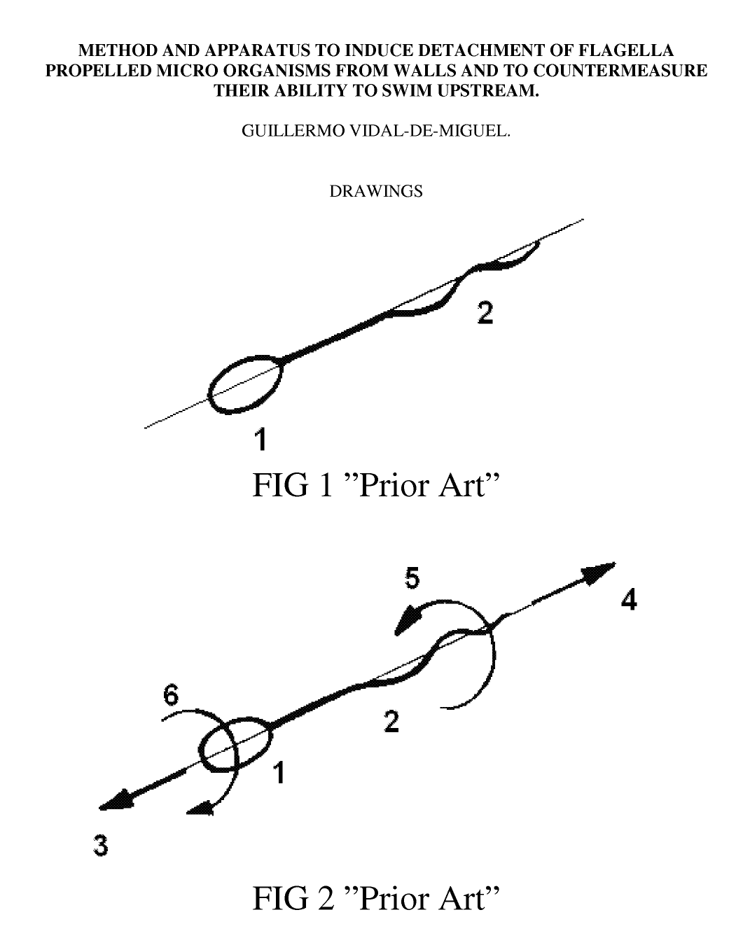

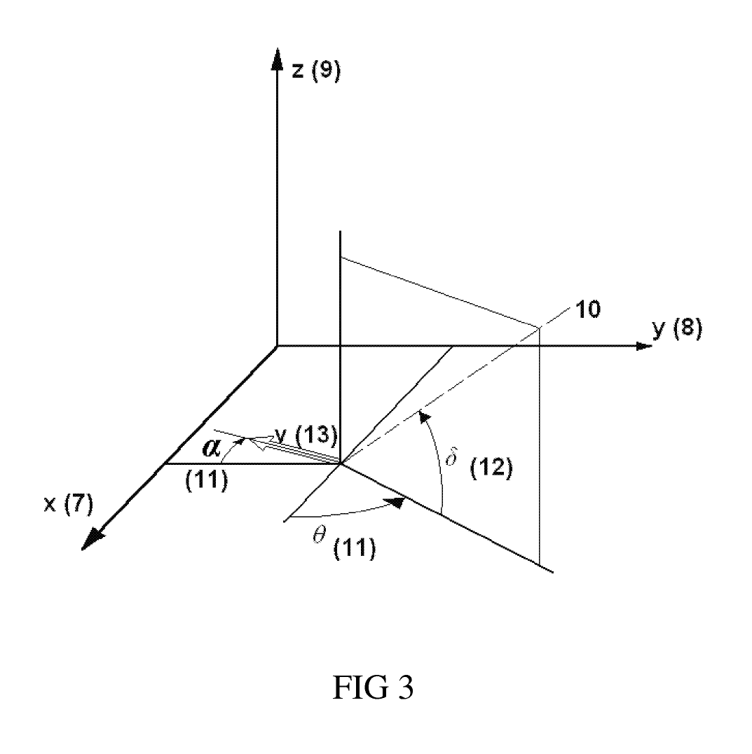

### A MORE DETAILED DESCRIPTION OF THE INVENTION

The purpose of the present invention is to find a solution to the related problem of (i) driving a fluid online through a duct form an origin to a destination while (ii) assuring that no microorganisms will swim upstream and also assuring that (iii) microorganisms and complex molecules traveling downstream with the fluid will not be destroyed, modified or killed and (iv) that the fluid chemical composition and properties will remain unchanged. To achieve this goal, the method has to be non aggressive with either the fluid nor the microorganisms and the complex molecules.

The present invention relies on the fluid mechanics configuration of the duct and on the FPMO swimming mechanics. The new specially designed configuration counter-measures mechanically the ability of FPMO to swim upstream.

It is interesting noting that, as described above, the FPMO requires both the proximity to a wall and a velocity gradient within a given range to swim upstream. Based on eliminating one or both of the ingredients needed by the FPMO, different strategies can be used to trip up the FPMO in its rush.

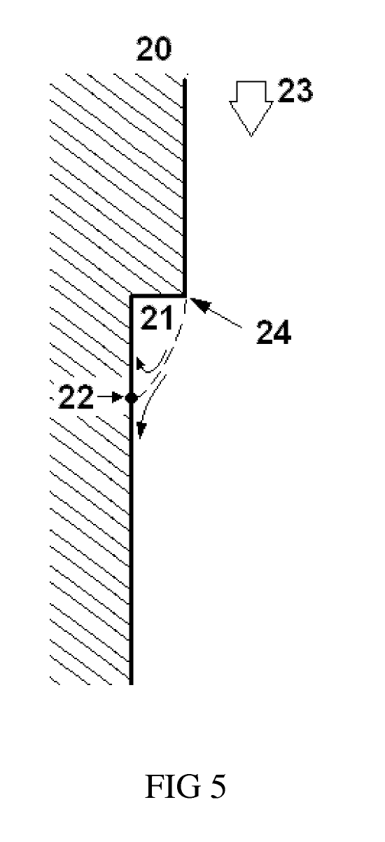

The first strategy proposed herein implies making a stagnation barrier; this could be done by means of a recirculation bubble. Said recirculation bubble would be annular in the case of a tube. FIG. 5 illustrates a longitudinal section of a specially designed wall of fluid duct (20) comprising a recirculation bubble (21) and a reattachment line (22). In this figure, the main direction of the fluid is represented by the arrow (23). The stagnation barrier is the reattachment line (22). Said reattachment line (22) would be annular in the case of a tube. Note that though the configuration shown in FIG. 5 produces the recirculation bubble by means of a square step (24), other configurations can easily produce similar recirculation bubbles and are also a part of this invention. Under the effect of the recirculation bubble, the FPMO would be always swimming towards the reattachment line since the flow near the wall seems to emerge from said line.

One problem of this solution is that FPMO would not be flushed and they would tend to accumulate in the reattachment line. Another problem of this strategy would be that the fluid configuration would become highly turbulent at high Reynolds numbers. For this reason, this configuration should be used preferably in small capillaries with low flow rates. The main advantage of this configuration is its simplicity which permits matching it in small capillaries for which it is suitable.

The second strategy proposed herein implies detaching the FPMO from the wall. Once the FPMO is detached from the wall, it will be flushed far downstream by the flow which is much stronger as the FPMO gets farther from the wall. Note that, due to the non slip boundary condition associated to the Navier-Stokes equations, the velocity profile tends to zero on the wall and increases farther from the wall while the velocity gradient also decreases as the FPMO is farther from the wall. Once the FPMO is detached, it also looses the torques and forces induced by the proximity of the wall. In the absence of those effects, the FPMO will no longer receive information about what is upstream and downstream. In this circumstance, the FPMO now swims freely as in unbounded fluid describing a straight trajectory in a random directions with respect to the fluid and is carried downstream by the fluid flow itself.

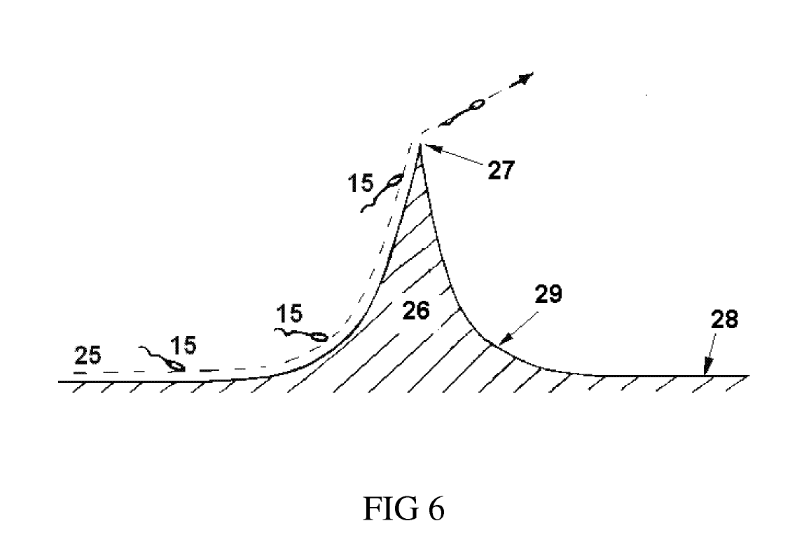

Detachment of the FPOM can be induced by means of a sharp edge. A FPMO sharp edge comprises a sharp edge and a smooth transition between the sharp edge and the original wall. FIG. 6 illustrates the trajectory (25) of an FPMO (15) when swimming over a FPMO detachment sharp edge (26). The FPMO can not change its swimming direction so abruptly to follow the geometry of the sharp edge (27), and therefore it detaches from the wall (28) at said sharp edge (27). In a real situation, ideal sharp edges do not exist. But the detachment is still produced if the radius of the tip is small enough. Detachment of FPMO from sharp corners or edges has already been proven satisfactorily by Peter Galagda et. al [7]. In their device, they use a set of funnels aligned to produce a wall that can be easily crossed in one direction but not in the opposite direction. Note that, though in the funnel designed by Galadja the FPMO detaches from the funnel, it is not intended to detach the FPMO from the main wall. In the design of the FPMO detachment sharp edge, it is also needed to avoid valley type edges that could trap the FPMO. For this reason, the transition region (29) between the original wall (28) and the sharp edge has to be smooth as shown in FIG. 6.

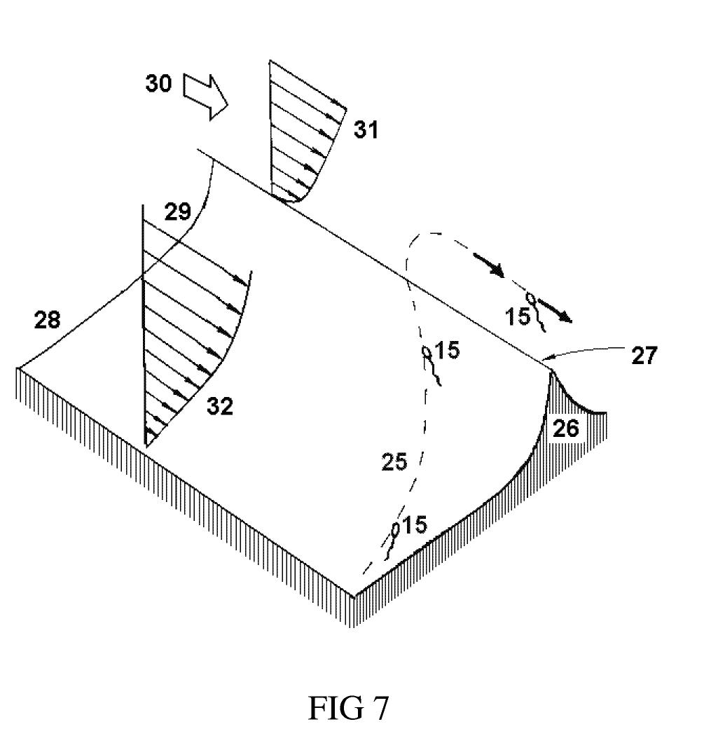

Hill et al. [2] have observed that velocity gradients higher than 10 s⁻¹ change the swimming configuration of E-Coli. High velocity gradients are expected to change the swimming configuration of all FPMO in the same way the affect E-Coli (Except because some FPMO rotate clockwise and other FPMO rotate counter-clockwise). At said high velocity gradients, the drag and torques induced by the velocity gradient will domain over all the rest of forces and torques. In this context, the FPMO will be aligned with the flow and dragged downstream. Under higher velocity gradients, the FPMO will be eventually detached from the wall as the forces that used to keep the FPMO attached to the wall become negligible when comparing with such a high velocity gradient. Said high velocity gradients can be produced by means of the sharp edge, preferably when said sharp edge is oriented with the same direction of the fluid velocity. If the sharp edge is aligned with the flow, then the fluid configuration can be considered to be governed by transversal stresses and the equations describing the velocity profile are the Poisson's equations. Poisson's equations produce singular results of its gradient near a sharp edge. This means that the velocity gradient tends to infinity near the sharp edge. This singular behavior also appears in the fluid boundary layer even when the regime implies higher Reynolds numbers and turbulent conditions. FIG. 7 illustrates the trajectory (25) of an FPMO (15) swimming over a FPMO detachment sharp edge (26) aligned with the flow velocity (30). The FPMO detaches from the sharp edge (27). FIG. 7 also represents the singular velocity profile (31) produced at the sharp edge (27) and the normal velocity profile (32) produced at any other non singular position of the wall (28) and the smooth transition region (29). Said singular behavior can also be achieved when the sharp edge is slightly misaligned with the flow. Though in the real world the ideal singularity can not be achieved, a very strong local velocity gradient will be achieved with an edge that is sharp enough. Said strong velocity gradient will have the same properties against FPMO as the ideal singularity.

The main new concept of this invention is using FPMO detachment sharp edges, preferably aligned with the flow. The FPMO detachment sharp edge shown in FIG. 6 is one of the preferred embodiment of the present invention. Other shapes of the FPMO detachment sharp edge will also induce FPMO detachment and high velocity gradients and they are therefore also a part of this invention. FPMO detachment can also be induced by FPMO detachment sharp edges non aligned with the fluid velocity, and they are also a part of this invention.

As the FPMO tends to remain in the vicinity of the wall due to the hydrodynamic trapping effect either with or without fluid velocity, the FPMO tend to accumulate in the proximity of the wall. This can be an advantage for the FPMO since this hydrodynamic trapping effect helps it to find its food and also helps it to be more pathogenic. Obviously this is in many applications a disadvantage for the humans. The use of FPMO detachment sharp edges with or without fluid velocity can be useful to avoid accumulation of FPMO on surfaces. This application of the FPMO detachment sharp edge is also incorporated in this invention.

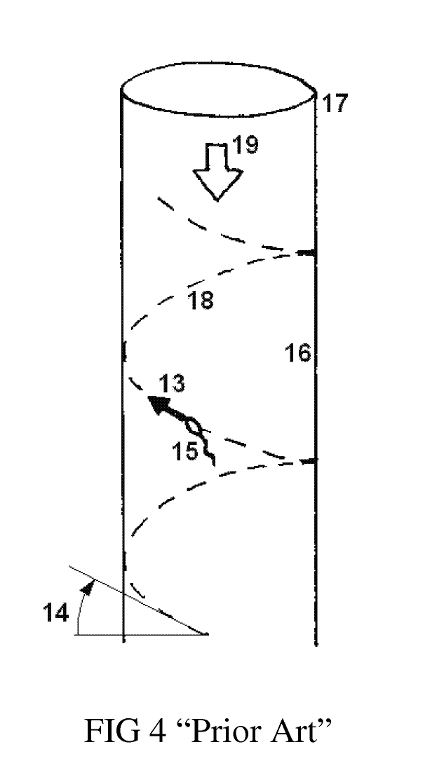

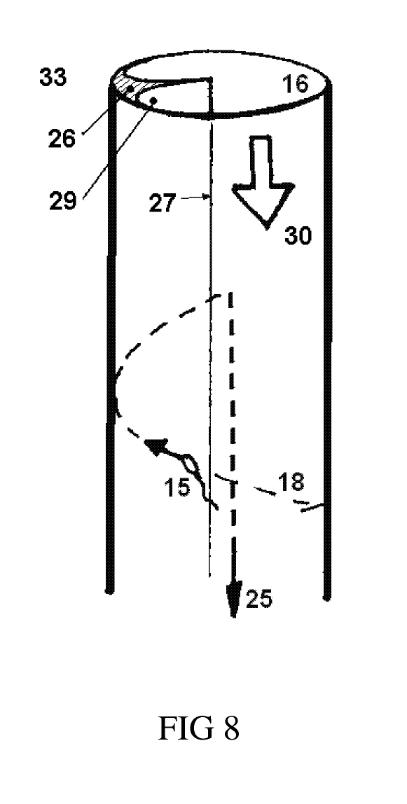

The typical shape of the trajectories described by a FPMO in a tube equipped with one of said FPMO detachment sharp edges is shown in FIG. 8. The direction of the fluid velocity is represented by the arrow (30). Once the FPMO (15) reaches the inner wall (16) of the specially designed tube (33), it starts its typical helical trajectory (18) beating upstream until, eventually, it reaches the FPMO detachment sharp edge (26). The smooth transition (29) drives the FPMO from the wall towards the sharp edge (27). In the sharp edge (27) the shape of the trajectory changes (25). The FPMO is detached from the wall and is flushed downstream by the strong local velocity gradient and the strong flow. As the trajectories are helical until the FPMO reaches The FPMO detachment sharp edge, the FPMO will find the FPMO detachment sharp edge before ending even one turnoff of the helix. The net effect is that the FPMO can not swim upstream further than a given maximum distance. Note the difference between the patterns shown in FIG. 7 and in FIG. 4 in which the FPMO can swim upstream endlessly.

As explained above, the use of FPMO detachment sharp edges can be also useful to countermeasure the ability of FPMO to swim upstream in any device. The preferred embodiment for this application is a tube comprising at least one FPMO detachment sharp edge, preferably aligned with the flow as described in FIG. 8. Said tube is herein termed FPMO detachment tube. Different sizes, shapes, number and distribution of FPMO detachment sharp edges can also be arranged in different tubes and they all are based on the same premises. Consequently, they are all FPMO detachment tubes and thus they are also a part of this invention. The FPMO detachment sharp edge can also be misaligned with the fluid flow in a FPMO detachment tube. For instance, it can be arranged in an inner helix like a thread inside the FPMO detachment tube.

FPMO detachment sharp edge can be used to counter-measure the ability of FPMO to swim upstream in any shape of tube and any other device different from a single circular tube, and its use in such devices is also a part of this invention.

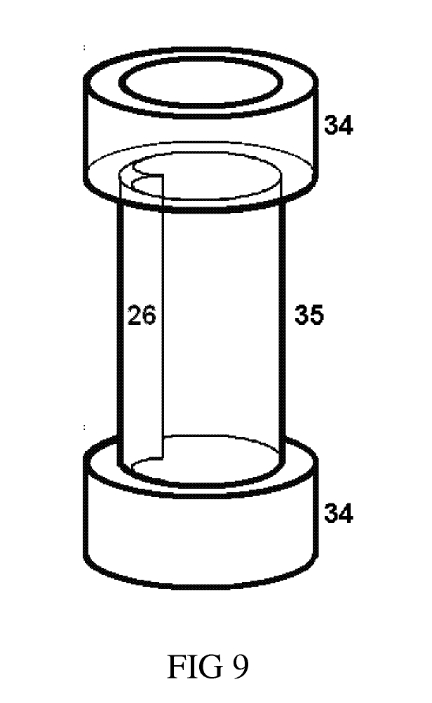

In many applications using rounded standard tube, such as medical devices or any other device comprising standard circular tube, it might be more convenient to use only a segment of the tube equipped with the FPMO detachment sharp edge. For those cases, a piece like that shown schematically in FIG. 9 comprising standard connections (34) to normal standard tube and the segment (35) equipped with FPMO detachment sharp edge (26) would be more advisable. Said piece is a FPMO detachment tube with standard fittings. This piece is also included in the present invention. Different configurations of said piece, comprising at least one FPMO detachment sharp edge are also included in this invention.

The FPMO detachment tube is especially useful to guarantee that microorganism can not swim upstream. Different practical uses of the FPMO detachment tube include driving fluids from clean systems to contaminated systems or sinks.

The FPMO detachment tube is useful in medical applications for humans or animals to reduce the probability of infections. For instance, normal catheter drainages and normal catheters are often necessary, especially after a surgery, and they can often produce infections as the FPMO can swim through them. In order to reduce the probability of infections, the FPMO detachment tube has to be used as catheter drainage or as a catheter in stead of the normal catheter drainages and normal catheter.

The FPMO detachment tube is also useful to assure that pathogens from an ill individual will not contaminate a system that is injecting a fluid in the body of said individual. In this case, the fluid has to be driven from the system to the individual through a FPMO detachment tube.

The FPMO detachment tube is also useful to introduce different flows online in FPMO cultures. If a FPMO detachment tube is used as a feeding probe. FPMO will not swim upstream and will not contaminate upstream the feeding probe.

The FPMO detachment tube can also be used to prevent contamination though downstream of the tube is also clean in normal operation. In this case, the FPMO detachment tube will act as an extra hygiene measure. The fluids have to be driven through said FPMO detachment tube. Its mission is preventing that the whole system is contaminated in case that the downstream part of the FPMO detachment tube gets contaminated by accident. This use of the FPMO detachment tube will be herein termed firewall FPMO detachment tube.

What is claimed:

1. An apparatus to detach flagella propelled microorganisms from the wall of a duct or of a container of liquid or of a solid immersed in said liquid, said flagella propelled microorganisms would otherwise be swimming in circles or heading upstream hydrodynamically trapped by said wall, said apparatus comprising:
(i) a sharp edge that emerges from said wall and
(ii) a smooth transition between said sharp edge and said wall.

2. An apparatus according to claim 1 where said sharp edge and said smooth transitions are shaped as in FIG. 6.

3. An apparatus according to claim 1 where detachment of said flagella propelled microorganism from the wall is induced at the sharp edge.

4. An apparatus to prevent said flagella propelled microorganisms from accumulating in said wall, according to claim 1, where said wall is equipped with at least one of said sharp edges and their smooth transitions, and where said flagella propelled microorganisms are detached from said wall.

5. An apparatus to counteract the ability of said flagella propelled microorganisms to swim upstream near a wall, according to claim 1, where the fluid is moving and said wall is equipped with at least one of said sharp edges and their smooth transitions, and where said flagella propelled microorganism is detached from said wall and flushed downstream by said moving fluid.

6. An apparatus to counteract the ability of said flagella propelled microorganisms to swim upstream through ducts, according to claim 5, where said wall is the inner surface of said duct.

7. An improved catheter for medical applications through which flagella propelled microorganisms are prevented from swimming upstream, according to claim 6, where said duct is used as a catheter.

8. The duct of claim 6 where said duct further comprises standard tube fittings.

9. The duct of claim 6 where said duct is used to drive fluids as a hygiene measure to prevent said flagella propelled microorganisms from spreading upstream of said ducts.

10. An apparatus to drag downstream with the moving fluid flagella propelled microorganisms hydrodynamically trapped by a wall, said flagella propelled microorganisms would otherwise be swimming heading upstream hydrodynamically trapped by said wall, said apparatus comprising:
(i) a sharp edge that emerges from said wall and
(ii) a smooth transition between said sharp edge and said wall.

11. An apparatus according to claim 10 where said sharp edge and said smooth transitions are shaped as in FIG. 6.

12. An apparatus according to claim 10 where fluid is moving and the velocity gradient produced at the sharp edge is higher than 5 s⁻¹.

13. An apparatus to counteract the ability of said flagella propelled microorganisms to swim upstream near a wall, according to claim 12, where said wall is equipped with at least one of said sharp edges and their smooth transitions, and where said flagella propelled microorganism is dragged downstream by said velocity gradient.

14. An apparatus to counteract the ability of said flagella propelled microorganisms to swim upstream through ducts, according to claim 13, where said wall is the inner surface of said duct.

15. An improved catheter for medical applications through which flagella propelled microorganisms are prevented from swimming upstream, according to claim 14, where said duct is used as a catheter.

16. The duct of claim 14 where said duct further comprises standard tube fittings.

17. The duct of claim 14 where said duct is used to drive fluids as a hygiene measure to prevent said flagella propelled microorganisms from spreading upstream of said ducts.

18. A method to countermeasure the ability of flagella propelled microorganisms from swimming upstream through ducts when they are hydrodynamically trapped by the wall, comprising producing a recirculation bubble in the fluid flow produced by an induced detachment of the flow followed by an induced reattachment of the flow in a reattachment line, said reattachment line acting as a barrier for said flagella propelled microorganisms that swim towards said reattachment line.
